(19) United States
(12) Plant Patent Application Publication
(10) Pub. No.: US 2019/0059183 P1
(43) Pub. Date: Feb. 21, 2019

(54) Kiwi plant named 'Cristal'

(50) ;

(71) Applicant: Gonzalo P. Espinosa Rodriguez, Curico (CL)

(72) Inventor: Gonzalo P. Espinosa Rodriguez, Curico (CL)

(21) Appl. No.: 15/731,859

(22) Filed: Aug. 15, 2017

(51) Int. Cl. A01H 6/00

(52) U.S. Cl. PLT/156

(57) ABSTRACT

A new and distinctive kiwi plant variety of the species Actinidia deliciosa, named ‘CRISTAL’ is described. The new variety is characterized by a larger and heavier fruit and lower acidity pulp than ‘Hayward’ variety. The hair of the fruit of ‘CRISTAL’ variety presents a weak adhesion to the epidermis, among other features. The shape and uniformity of the fruit facilitates its packing.

Latin name of the genus and species: Actinidia deliciosa.

Variety denomination: ‘CRISTAL’.

### BACKGROUND

Cultivated kiwi plants are dioecious deciduous vines of Actinidia deliciosa. The genus Actinidia is originated from China and parts of Asia. Nowadays several species are cultivated outside their native range and known varieties or cultivars of Kiwi plants are commercially grown in many countries including Chile.

### SUMMARY OF THE INVENTION

The present invention is a new and distinct variety of Actinidia deliciosa named ‘CRISTAL’. It corresponds to the finding of a new and distinguishing adult plant in a ‘Hayward’ orchard, located in Parcela No. 36, Los Cristales, Curico, Chile. The new variety has been asexually reproduced due to its several advantageous characteristics from other kiwi plants.

The new variety is characterized by a larger and heavier fruit, sweeter and lower acidity pulp than ‘Hayward’ variety, and less seeds than ‘Hayward’. The hair of the fruit of kiwi ‘CRISTAL’ variety presents a weak adhesion to the epidermis, among other features. The shape and uniformity of the fruit facilitates its packing.

### BRIEF DESCRIPTION OF THE PHOTOGRAPHS

The accompanying photographs in full color illustrate the new kiwi plant ‘CRISTAL’ variety as well as comparisons with ‘Hayward’ and ‘Bruno’ varieties.

FIG. 1 illustrates examples of typical fruit harvested from ‘CRISTAL’.

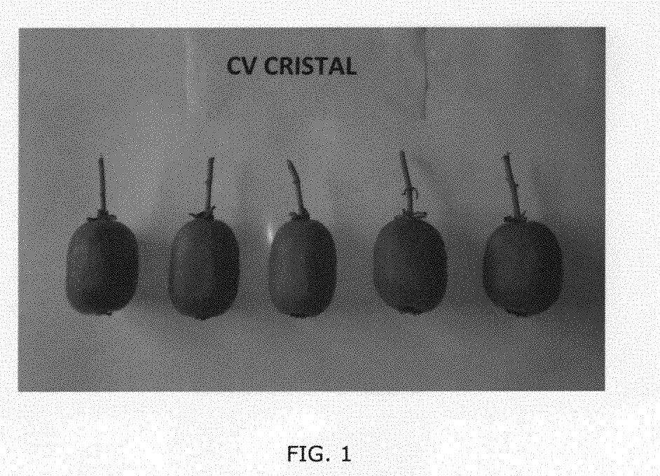

FIG. 2 illustrates a typical fruit of ‘CRISTAL’ as compared to a typical fruit of ‘Hayward’ and a typical fruit of ‘Bruno’.

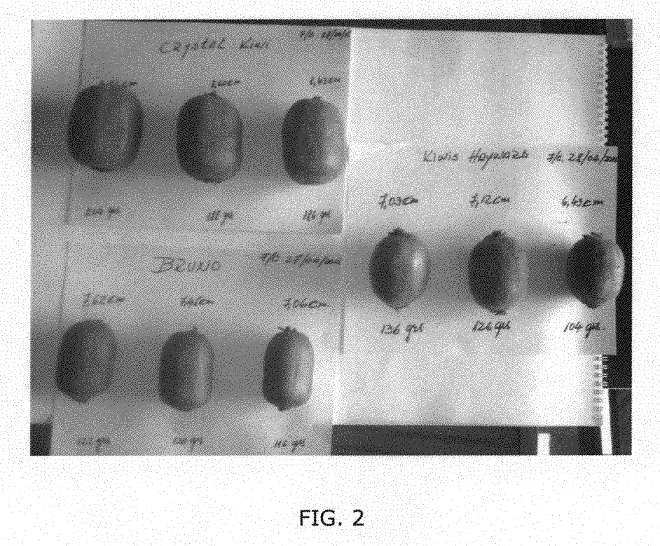

FIG. 3 illustrates a cross-section and a side view of a typical fruit of ‘CRISTAL’ as compared to a typical fruit of ‘Hayward’.

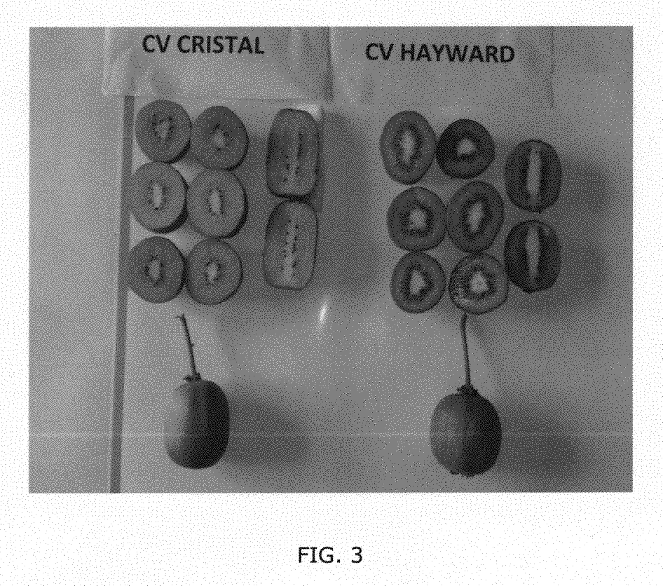

FIG. 4 illustrates typical flowers of ‘CRISTAL’.

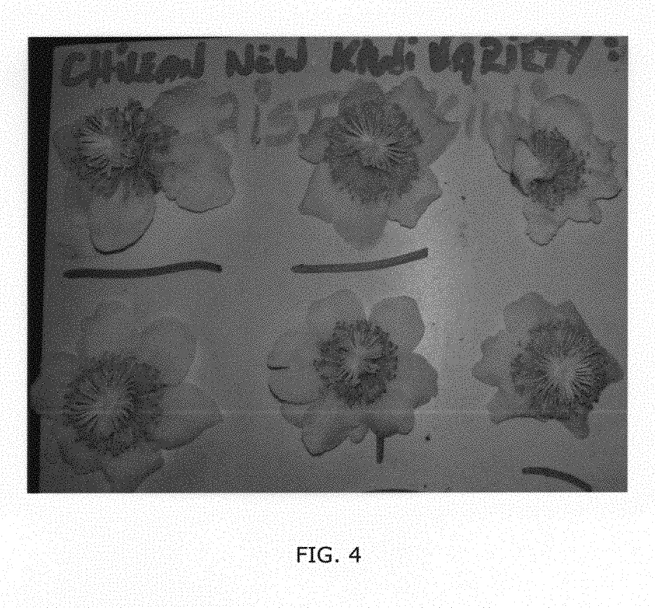

FIG. 5 illustrates typical foliage, hanging fruit and stems fruit of ‘CRISTAL’ on the vine.

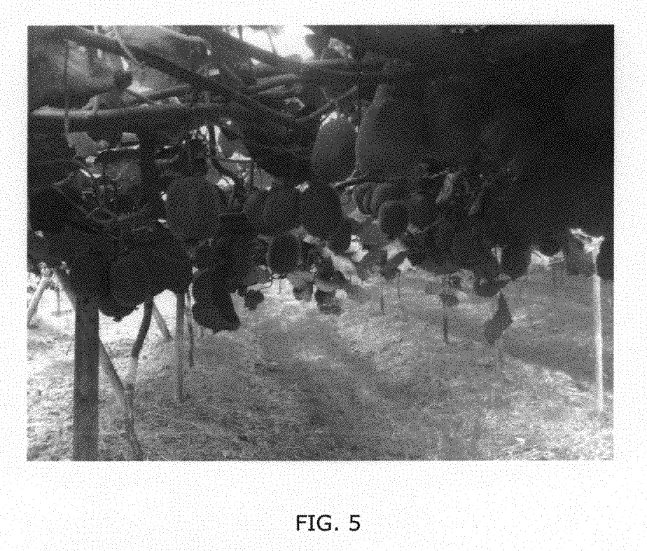

Conventional techniques were used for capturing images/photographs. It might occur that colors may appear different from actual colors, due to light reflectance; however the images material provided are as accurate as possible by the used conventional photographic techniques.

### DETAILED DESCRIPTION OF THE INVENTION

The present invention is a new and distinct variety of Actinidia deliciosa named ‘CRISTAL’ which has several advantageous characteristics from other kiwi plants, as mentioned above.

The new Kiwi plant variety corresponds to the finding of a new and distinguishing plant in a ‘Hayward’ orchard.

The mother plant of kiwi ‘CRISTAL’ variety was discovered in 2001, by simple observation. The discovered plant fruit corresponded to an adult plant present in an orchard established in 1987. The plant showed a high fruit yield, with larger size and a desirable shape for packaging purposes. During 2010 the new plant began to be asexually propagated, establishing a first orchard of 79 plants, which were vegetatively propagated and grafted on ‘Hayward’ rootstocks.

If compared the kiwi ‘CRISTAL’ variety versus ‘Hayward’, both the plant and the fruit are different: the ‘CRISTAL’ fruit is heavier. In addition, a kiwi ‘CRISTAL’ plant yields more units of fruit than a ‘Hayward’ plant. Both characteristics result in that the production of kiwi ‘CRISTAL’ variety is almost double than ‘Hayward’.

Other differences with ‘Hayward’ are that kiwi ‘CRISTAL’ variety has a creamy green color and its flavor is sweeter and aromatic.

The new kiwi plant ‘CRISTAL’ variety has been continuously asexually reproduced by cuttings from 2010 on. The distinguishing characteristics of the variety have been stably maintained in the propagated plants, as was shown by 79 plants, from which more than 2,300 new plants, covering about 3-4 hectares, have been propagated during 2017. The new variety has been asexually reproduced by grafting into rootstocks of ‘Hayward’.

Male pollinizers suitable for kiwi plant ‘CRISTAL’ variety may include, but are not limited to ‘Matua’.

### Kiwi ‘CRISTAL’ Variety Characteristics

The following table (Table 1) describes the main physical characteristics of Kiwi plant ‘CRISTAL’ variety. The Horticultural terminology applied below is used in accordance with UPOV guidelines for kiwi (Test Guidelines—TG/98/6—Actinidia 2001-04-04, available at http://www.upov.int/en/publications/tg-rom/tg098/tg_98_6. pdf).

TABLE 1

| UPOV No. | UPOV Characteristics for Kiwi | ‘CRISTAL’ |
|---|---|---|
| 4 | Plant: vigor | Strong |
| 26 | Leaf blade: shape | Broad ovate |
| 27 | Leaf blade: shape of apex | Acute |
| 32 | Leaf blade: green color of upper side | Light |
| 51 | Petal: curvature of apex | Strongly expressed |
| 63 | Fruit: size | Very large |
| 64 | Fruit: general shape | Oblong |
| 66 | Fruit: general shape of stylar end | Slightly depressed |
| 68 | Fruit: shape of shoulder at stalk end | Squared |
| 76 | Fruit: type of hairiness | Hirsute |
| 91 | Time of beginning of flowering | Late |

Table 2 provides the features according to the Table of characteristics for Actinidia (Test Guidelines—TG/98/6—Actinidia 2001-04-04, see above) defined by UPOV for Kiwi ‘CRISTAL’ variety. The UPOV characteristics correspond to botanical terms known in the art for description of plant varieties.

TABLE 2

| UPOV No. | Characteristics | State of expression |
|---|---|---|
| 5 | Young shoot: Hairiness | Present |
| 6 | Young shoot: Density of hair | Medium |
| 7 | Young shoot: Type of hairiness | Hirsute |
| 8 | Young shoot: Anthocyanin coloration | Strong |
| | of growing tip | |
| 9 | Stem: Thickness | Medium |
| 11 | Stem: Roughnes of bark | Rough |
| 12 | Stem: Hairiness | Present |
| 13 | Stem: Density of hair | Medium |
| 14 | Stem: Type of hairiness | Bristly |
| 15 | Stem: Size of lenticels | Small |
| 16 | Stem: Number of lenticels | Few |
| 19 | Stem: Size of bud support | Small to medium |
| 20 | Stem: Profile of proximal face of bud | Straight |
| | support (if sloping) | |
| 21 | Stem: Presence of bud cover | Absent |
| 22 | Stem: Size of hole in bud cover | Small |
| 23 | Stem: Leaf sear | Shallow |
| 26 | Leaf blade: Shape | Broad ovate |
| 27 | Leaf blade: Shape of apex | Acute |
| 28 | Leaf blade: Arrangement of basal lobes | Slightly apart |
| 29 | Leaf blade: Hair on upper side | Absent or very |
| | | sparse |
| 30 | Leaf blade: Hair on lower side | Medium |
| 31 | Leaf blade: Puckering/Blistering on | Medium |
| | upper side | |
| 34 | Leaf blade: Presence of variegation | Absent |
| 37 | Leaf: Ratio petiole length/blade length | Absent |
| 38 | Petiole: Density of hair | Dense |
| 39 | Petiole: Anthocyanin coloration on | Weak |
| | tipper side | |
| 40 | Flower bud: anthocyanin coloration | Medium |
| | of protruding petal end | |
| 41 | Inflorescence: Predominant number | 1 |
| | of flowers | |
| 42 | Flower stalk: Length | Long |
| 43 | Flower stalk: Density of hair | Dense |
| 44 | Flower stalk: Length of hair | Medium |
| 45 | Flower: Number of sepals | >5 |
| 47 | Sepal: Density of hair | Sparse |
| 48 | Sepal: Length of hair | Short |
| 49 | Flower: Diameter | Very large |
| 50 | Flower: Arrangement of petals (viewed | Overlapping |
| | from beneath) | |
| 51 | Petal: Curvature of apex | Strongly expressed |
| 52 | Petal: Type of coloration (adaxial side) | Bicolored |
| 54 | Petal: Different shades of color | Present |
| 60 | Styles: Number | Many |
| 62 | Styles: Attitude | Horizontal |
| 63 | Fruit: Size | Very large |
| 64 | Fruit: General shape | Oblong |
| 65 | Fruit: Shape in cross-section (at median) | Oblate |
| 66 | Fruit: General shape at stylar end | Slightly depressed |
| 67 | Fruit: Presence of calyx ring | Weakly expressed |
| 68 | Fruit: Shape of shoulder at stalk end | Squared |
| 69 | Fruit: Length of stalk | Long |
| 70 | Fruit: Ratio stalk length/fruit length | Large |
| 71 | Fruit: Persistence of sepals | Present |
| 72 | Fruit: Conspicuousness of lenticels on | Inconspicuous |
| | skin | |
| 74 | Fruit: Hairiness of skin | Absent |
| 75 | Fruit: Density of hair | Medium |
| 76 | Fruit: Type of hairiness | Hirsute |
| 77 | Fruit: Distribution of hairs | Evenly spread |
| 79 | Fruit: Adherence of hair to skin (when | Weak |
| | rubbed) | |
| 84 | Fruit: Diameter of core relative to fruit | Large |
| 85 | Fruit: General shape of core (in cross | Transverse elliptic |
| | section) | |
| 86 | Fruit: Fluting of core (in cross section) | Present |
| 88 | Fruit: Sweetness | Medium |
| 89 | Fruit: Acidity | Medium |
| 90 | Time of vegetative bud burst | Late |
| 91 | Time of beginning flowering | Late |
| 92 | Time of maturity for harvest | Late |

Further, the following measurements are typical of kiwi ‘CRISTAL’ variety, these are all data collected from the 79 plants established in 2010:

### Plant

The average height of the ‘CRISTAL’ plant is 1.9 m over vine structure. The plant has a stem diameter of 12 mm when reaching 1 meter of the yearly growth (50% of the year total sucker length). The stem length can be 2-2.8 m or more. The average trunk diameter is 100 mm (height=1 m). The average trunk length is 1.65 m up to the origin of the first arm. The trunk texture has stripes in bark and the natural peeling is less than ‘Hayward’.

The stem has a low number of lenticels, similar to ‘Meteor’, arranged all over the surface, in disorder, not displayed in straight lines, of brownish color. The shape of the lenticels is elongated ovoid, very few are rounded. The lenticels have an average length of 0.5-1 mm, width of 0.3-0.5 mm and a minimum density of 5-6 per cm².

### Foliage

The leaf of kiwi ‘CRISTAL’ variety is large, dark green, pointed like ‘Tomuri’, smaller if compared to ‘Hayward’. The leaf average length is 160 mm, and the leaf average length to the petiole cavity, which is an open cavity, is 150 mm to the end of the midrib. The leaf average width is 170 mm. The leaf shape is round, with an average apical protuberance of 15 mm. The average leaf apex is 15 mm length. The leaf base is open in the petiole cavity. The leaf union is petiolated, with an average petiole length of 85 mm. The leaf margin is ciliated, with cilia separated by 2-3 mm. The leaf upper surface texture is a smooth layer and the lower surface has cotton-like texture.

### Flowers

The typical flower of kiwi ‘CRISTAL’ variety has 6 sepals, displaying 6 to 7 petals. The bud color is beige. The bud opening is 4-6 days after ‘Hayward’. The flower diameter is very large, same as ‘Hayward’. The flower depth is shallow, with petals curved towards the lower part (towards the stalk). The flower has overlapping petals. The petals color is yellowish white. The style attitude is horizontal, similar to ‘Bruno’. The flower has a high number of styles. The ovary color is greenish white. The number of sepals is higher than 5. Further in kiwi ‘CRISTAL’ variety, flowers are convex, whereas in ‘Hayward’ flowers are concave.

### Fruit

The fruit average weight of kiwi variety ‘CRISTAL’ is 180 g. The average length is 70-75 mm. The average diameter is 50 mm×60 mm. The core diameter is 20 mm×35 mm. The general shape is ovoid, elongated. The cross-section at median is slightly oval-shaped. The stylar end shape is pointed. The shoulder shape is oval-shaped. The calyx ring is present, large and sunken, exhibiting few seeds. The calyx ring expression is weak. The skin at harvest is light brown. The skin hairiness of the fruit is medium. The hairiness adherence on the fruit skin is weak. The fruit core is oval-shaped. The fruit contains lenticels in high density, with a size of 0.3 mm, separated by 0.3 mm; the number of locules per fruit is 33-35. The length of locules is 4 mm, with a width of 1-1.25 mm. The ripe seed color is black. The withered seed color is dark brown. The fruit stem has a length of 50-65 mm, a diameter of 2.5-3.5 mm and a smooth, cotton-like texture.

The fruit stalk has a length of 50-60 mm, a diameter of 2-3 mm and displays a rigid, semi-hard texture.

Table 3 provides the color description is for Kiwi ‘CRISTAL’ variety according to UPOV descriptors.

TABLE 3

| UPOV No. | Characteristics | Color |
|---|---|---|
| 10 | Stem: Color of shoot on sunny side | Red brown |
| 17 | Stem: Color of lenticels | Brownish |
| 32 | Leaf blade: Color of upper side | Light |
| 33 | Leaf blade: Color of lower side | Light green |
| 46 | Sepal: General color | Brown |
| 53 | Petal: Main color on adaxial side | Yellowish white |
| 58 | Filament: Color | White |
| 59 | Anther: Color | Yellow |
| 61 | Styles: Color | Whitish yellow |
| 73 | Fruit: Color of skin | Greenish brown |
| 78 | Fruit: Color of hairs | Dark brown |
| 80 | Fruit: Color of skin at | Light brown |
| | maturity for consumption | |
| 82 | Fruit: Color of outer pericarp | Medium green |
| 83 | Fruit: Color of inner pericarp | Medium green |
| 87 | Fruit: Color of core | Greenish white |

### Other Characteristics

This new variety has been assayed for Pseudomonas syringae pv. actinidiae (Psa) presence and it is free of this pathogen.

What is claimed is:

1. A new and distinct variety of Actinidia deliciosa plant named ‘CRISTAL’ as herein illustrated and described.
